(19) United States
(12) Patent Application Publication
(10) Pub. No.: US 2024/0412846 A1
(43) Pub. Date: Dec. 12, 2024

(54) Systems and Methods for Facilitating and Evaluating Psychological Shifts in Users

(71) Applicant: Soul Studio LLC, New York, NY (US)

(72) Inventor: Abhay Mohan Wadhwa, New York, NY (US)

(21) Appl. No.: 18/332,634

(22) Filed: Jun. 9, 2023

(51) Int. Cl. G16H 20/70; A61B 5/16; G16H 50/20

(52) U.S. Cl. CPC G16H 20/70; A61B 5/165; G16H 50/20

(57) ABSTRACT

A system and method that provides a technical solution to the existing problem of deficient technologies in psychological evaluation. By utilizing a combination of hardware components and algorithms, the system accurately calculates the psychological shift in an individual at any given time. The system uses color options determined by a color psychology model and novel formulas derived from a psychological evaluation model to calculate mental parameters such as the Alignment Index, Conditioning Index, Dispersion Index, and Confusion Index. The system can include a user interface and can integrate with additional devices, such as biometric sensors, to enhance evaluation. The disclosed system is a comprehensive approach to psychological evaluation and monitoring, empowering individuals to proactively manage their mental health.

### FIELD OF THE DISCLOSURE

The present disclosure relates generally to the field of data processing. More specifically, the present disclosure is systems and methods for facilitating evaluating psychological shifts in users.

### TECHNICAL PROBLEM

As an individual grows up, it is very normal for the individual's mentality to change with time mainly due to social conditioning or under influence of some other individuals which could be contrary to the individual's optimum mental state.

Some systems take the birth data of an individual as input in the initial part of the method to calculate some values to define the characteristics of the individual and enable the individual to select colors from an ordinary set of colors at any point of time and conduct an assessment for providing an interpretation of the color selection in the form of the emotion of the individual.

Existing techniques for the interpretation of the color selection in the form of the emotion of the individual are deficient with regard to several aspects. For instance, current technologies do not calculate a psychological shift in the individual from the ideal psychological state of the individual. Furthermore, current technologies do not determine the ideal psychological state of the individual. Moreover, current technologies do not assign values to the selection of colors to calculate the psychological shift.

Therefore, there is a need for systems and methods for facilitating evaluating psychological shifts in users that may overcome one or more of the above-mentioned problems and/or limitations.

### TECHNICAL SOLUTION

The disclosed system and method offer a technical solution to the existing problem of deficient technologies that fail to evaluate psychological shifts to determine the ideal psychological state of an individual.

By utilizing a communication device, a processing device, and a storage device, the system and method can accurately calculate the psychological shift in an individual at any given time using color options determined by a color psychology model and novel formulas derived from a psychological evaluation model. The system and method assign numerical values to the color selections made by the user and analyze the data to determine mental parameters such as, but not limited to, the Alignment Index, Conditioning Index, Dispersion Index, and Confusion Index.

The combination of hardware components and algorithms provides a comprehensive approach to psychological evaluation and monitoring, enabling users to proactively manage their mental health. The system and method include a user interface and can integrate with additional devices, such as biometric sensors, to enhance evaluation. By addressing the existing problems in the field of psychological evaluation, the present disclosure provides a technical solution to individuals seeking to maintain a balanced and healthy mental state.

### SUMMARY OF THE DISCLOSURE

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

According to some embodiments, a method for facilitating evaluating psychological shifts in users is disclosed. Accordingly, the method may include receiving, using a communication device, a request associated with at least one user from at least one user device. Further, the method may include a step of retrieving, using a storage device, at least one ideal value corresponding to an ideal psychological state of the at least one user based on the request. Further, the method may include a step of transmitting, using the communication device, one of at least one instruction and one of a plurality of color options corresponding to one of the at least one instruction to the at least one user device. Further, the method may include a step of receiving, using the communication device, one of at least one selection of at least one color option from one of the plurality of color options corresponding to one of the at least one question from the at least one user device. Further, the method may include a step of determining, using a processing device, one of at least one value corresponding to one of the at least one selection of the at least one color option based on one of the at least one selection of the at least one color option. Further, the method may include a step of analyzing, using the processing device, one of the at least one value and the at least one ideal value based on at least one formula. Further, the method may include a step of calculating, using the processing device, at least one mental parameter of the at least one user based on the analyzing of one of the at least one value and the at least one ideal value. Further, the method may include a step of determining, using the processing device, a psychological shift in the at least one user from the ideal psychological state based on the at least one mental parameter. Further, the method may include a step of generating, using the processing device, a notification of the psychological shift of the at least one user based on the determining of the psychological shift. Further, the method may include a step of transmitting, using the communication device, the notification to the at least one user device.

According to some aspects, a system for facilitating evaluating psychological shifts in users is disclosed. Accordingly, the system may include a communication device, a processing device, and a storage device. Further, the communication device may be configured for receiving a request associated with at least one user from at least one user device. Further, the communication device may be configured for transmitting one of at least one instruction and one of a plurality of color options corresponding to one of the at least one instruction to the at least one user device. Further, the communication device may be configured for receiving one of at least one selection of at least one color option from one of the plurality of color options corresponding to one of the at least one question from the at least one user device. Further, the communication device may be configured for transmitting a notification to the at least one user device. Further, the processing device may be communicatively coupled with the communication device. Further, the processing device may be configured for determining one of at least one value corresponding to one of the at least one selection of the at least one color option based on one of the at least one selection of the at least one color option. Further, the processing device may be configured for analyzing one of the at least one value and at least one ideal value based on at least one formula. Further, the processing device may be configured for calculating at least one mental parameter of the at least one user based on the analyzing of one of the at least one value and the at least one ideal value. Further, the processing device may be configured for determining a psychological shift in the at least one user from the ideal psychological state based on the at least one mental parameter. Further, the processing device may be configured for generating the notification of the psychological shift of the at least one user based on the determining of the psychological shift. Further, the storage device may be communicatively coupled with the processing device. Further, the storage device may be configured for retrieving the at least one ideal value corresponding to an ideal psychological state of the at least one user based on the request.

In some examples, the system can comprise a communication device, such as a smartphone, tablet, computer, or wearable device, that uses a color psychology model to determine a plurality of color options. The system may also include instructions that assess specific aspects of the user's psychological state, derived from a psychological evaluation model, and machine learning algorithms that analyze user data and historical data to determine the psychological shift.

Additionally, the system can also allow the user to set personal goals, provides recommendations for actions to address psychological shifts, and generates periodic assessments and notifications. The communication device may also have a user interface that allows the user to interact with the system and input data, while biometric sensors and other devices can enhance evaluation. The system can also provide anonymized data to researchers or healthcare providers and generate visual representations of the user's psychological state. The storage device may also store data related to the user's mental well-being to provide a comprehensive overview.

In some examples, the system is designed to compare the user's mental parameters to a normative database of mental parameters, allowing for an accurate analysis of the user's psychological state. In certain implementations, the system also provides personalized feedback to the user based on their individual psychological state and ideal values, empowering them to take action to address any psychological shifts. The system may also provide periodic assessments and notifications to track the user's psychological state over time, enabling them to monitor their progress towards personal goals related to their mental well-being. The system may also aim to contribute to the improvement of mental health research and understanding by providing anonymized data to researchers and healthcare providers.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a system for facilitating evaluating psychological shifts in users, in accordance with some embodiments.

FIG. 3 is a flowchart of a method for facilitating evaluating psychological shifts in users, in accordance with some embodiments.

FIG. 4 is a flowchart of a method of determining ideal values of ideal psychological states of the users for facilitating the evaluating of the psychological shifts in the users, in accordance with some embodiments.

FIG. 5 illustrates a graphical interface screen for receiving a selection for a variable A for facilitating the evaluating of the psychological shifts in the users, in accordance with some embodiments.

FIG. 6 illustrates a graphical interface screen for receiving a selection for a variable B for facilitating the evaluating of the psychological shifts in the users, in accordance with some embodiments.

FIG. 7 illustrates a graphical interface screen for receiving a selection for a variable D for facilitating the evaluating of the psychological shifts in the users, in accordance with some embodiments.

FIG. 8 illustrates a graphical interface screen for receiving a selection for a variable E for facilitating the evaluating of the psychological shifts in the users, in accordance with some embodiments.

FIG. 9 illustrates a graphical interface screen for receiving a selection for a variable A1 for facilitating the evaluating of the psychological shifts in the users, in accordance with some embodiments.

FIG. 10 is a table for values for selections of variables for facilitating the evaluating of the psychological shifts in the users, in accordance with some embodiments.

FIG. 11 is a table for values for selections of other variables (variable A1) for facilitating the evaluating of the psychological shifts in the users, in accordance with some embodiments.

FIG. 12 is a block diagram of a computing device for implementing the methods disclosed herein, in accordance with some embodiments.

### DETAILED DESCRIPTION OF THE DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein-as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of systems and methods for facilitating evaluating psychological shifts in users, embodiments of the present disclosure are not limited to use only in this context.

### Overview

The present disclosure describes systems and methods for facilitating evaluating psychological shifts in users.

Further, the present disclosure describes the correlation of the color selection on any particular day with the birth data to calculate the psychological shift (change in a person's mental state as compared to their Optimum Mental State) in a person at any given point of time.

Taking input as one or more colors selected by the user and assigning those colors a numerical value and the assigning the numerical value to the colors are generalized, for eg: 1—red, 2—orange, 3—yellow, 4—green, 5—blue, 6—blue, 7—violet, 8—pink, 9—gold, hence, this assigning numerical values to colors should be further worked upon for advanced and accurate evaluation of the psychological state of the user.

There is no tool or mechanism available which is capable of calculating various emotive parameters and accordingly evaluating the psychological shift or deviation in a user. Further, the present disclosure describes that the shift is evaluated with respect to the ideal psychological state which is calculated corresponding to the birth details.

Further, the present disclosure describes the calculating of the person's optimum mental state. In order to calculate the person's optimum mental state, the epistemic number is calculated by adding up digits of the birth date and further reduced to a single digit, which is equivalent to his epistemic number if the person is born after 4 AM, and if the person is born before 4 AM, the calculated epistemic number is subtracted by one to calculate the actual epistemic value for the person.

Further, the present disclosure describes a system and method for evaluating the psychological shift of the person at any point of time by calculating mental parameters such as coefficient of Alignment index, Conditioning index, Dispersion Index, and Confusion index. These parameters are calculated based on the current color choices relative to the actual epistemic and destiny number of the person, wherein each color available in the system is assigned a specific numerical value. The other variables that play a major role in calculating the above parameters are most favorite color, positive affirmation, least favorite color, negative affirmation, person's mood, art selection, activity selection, psychological profile, and Vritti, wherein each such variable is designated a numerical value which is compared to values corresponding to the birth data (epistemic number and destiny number) to calculate the mental shift of the person.

The definition of the mental parameters is as follows:
1. Optimum Mental State: This is the psychological state of a person that is ideal for their overall positive progress. It refers to a mental state that allows an individual to respond effectively to life situations without being affected by high emotional arousal or similar personality patterns.
2. Alignment Index: A person's Alignment Index measures the deviation of their positive affirmation mental state from their Optimum Mental State. This index provides insight into how life situations have affected a person's current positive mindset. A lower index value indicates a more balanced and better-aligned mental state.
3. Conditioning Index: A person's Conditioning Index measures the deviation of their positive choices or likes from their Optimum Mental State. It assesses the impact of life situations on a person's overall positive mindset. A lower index value signifies a more balanced and better-aligned mental state.
4. Dispersion Index: A person's Dispersion Index measures the deviation of a shift in their positive choices or likes from their positive affirmations mental state. This index helps to evaluate how life situations have influenced a person's positive mindset and positive affirmations. A lower index value denotes a more balanced and better-aligned mental state.
5. Confusion Index: A person's Confusion Index measures the deviation of a shift in their negative choices or dislikes from their negative affirmations mental state. This index offers insight into how life situations have impacted a person's mindset regarding their dislikes. A lower index value indicates a less confused mental state.
6. Affirmation Index: A person's Affirmation Index measures the deviation of a shift in their negative choices or dislikes from their positive affirmations mental state. This index evaluates the impact of life situations on a person's relationship between positive and negative affirmations. A higher index value represents a more balanced mental state.

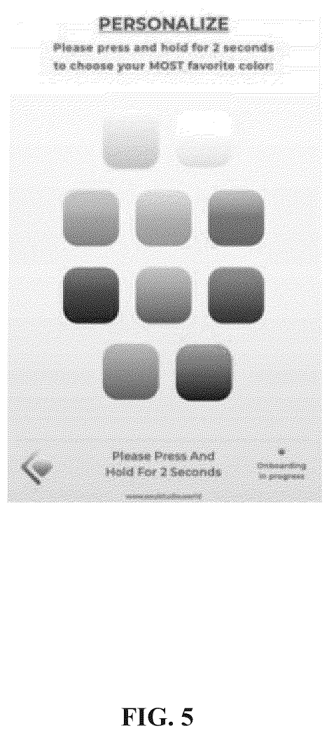

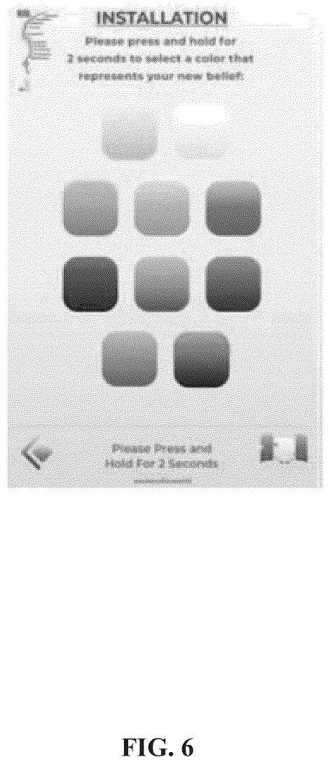

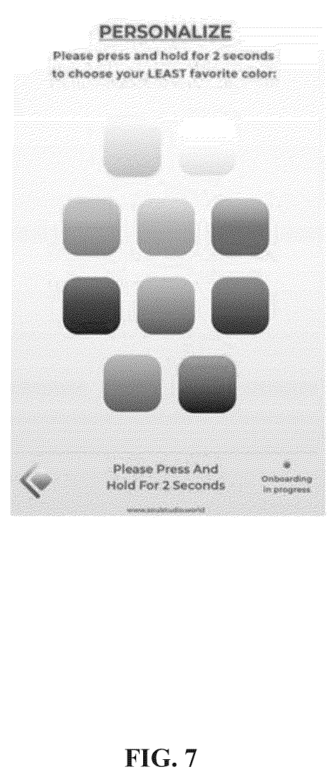

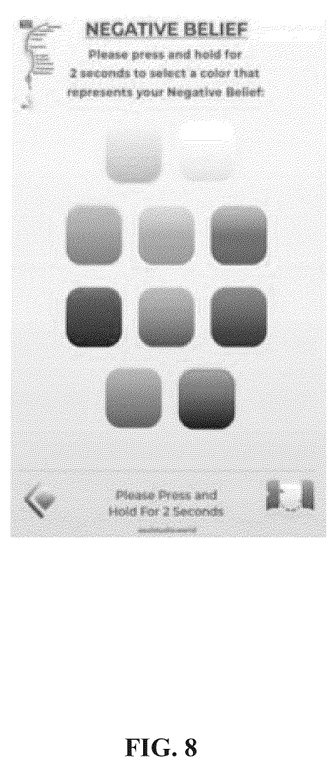

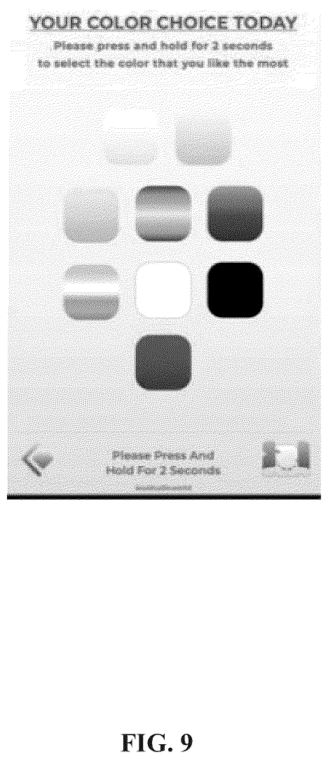

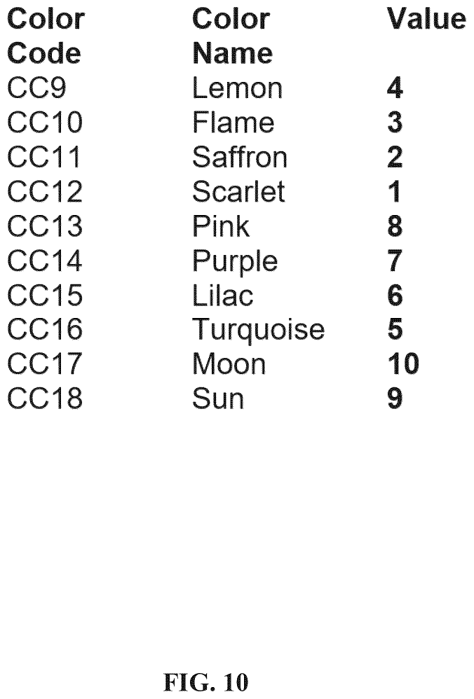

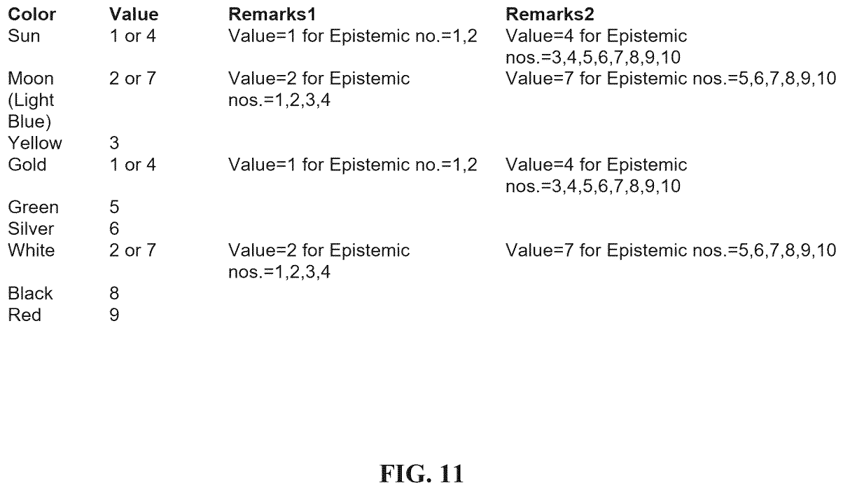

In order to calculate the above Indexes, the following are the variables, generated from user data and input:
A. Most Favorite Color (Variable A) INPUT=User Selection from the Graphic Interface Screen (see FIG. 5) This is then converted to a corresponding number (see FIG. 10).
B. Positive Affirmation (Variable B) INPUT=User Selection from the Graphic Interface Screen (see FIGS. 6). This is then converted to a corresponding number (see FIG. 10).
C. Destiny Number (Variable C) INPUT=Date of Birth (in DDMMYYYY format). Destiny Number is the Digital Root of the DOB (range—from 1 to 9).
D. Least Favorite Color (Variable D) INPUT=User Selection from the Graphic Interface Screen (see FIG. 7) This is then converted to a corresponding number (FIG. 10).
E. Negative Affirmation (Variable E) INPUT=User Selection from the Graphic Interface Screen (see FIG. 8) This is then converted to a corresponding number (see FIG. 10).
F. User ‘Color Choice Today’ (Variable A1) INPUT=User Selection from the Graphic Interface Screen (see FIG. 9) This is then converted to a corresponding number (FIG. 11).
G. Epistemic Number (Variable B1) INPUT=Day of Birth (in DD format). The Epistemic Number is the Digital Root of the DD above (range—from 1 to 9).
H. Name-Color/Number
I. Mood Profile-User Selection (only stored into User Data currently)
J. Art Profile-User Selection (only stored into User Data currently)
K. Activity Profile-User Selection (only stored into User Data currently)
L. Psychological Profile (Dosha, Kosha)
M. Vritti Profile

Further, an Alignment Index is a shift in one's current Positive Affirmation and his Destiny number. Conditioning Index is the shift in one's current Most Favorite Color and his Destiny number. Dispersion Index is the shift in one's current Most Favorite Color and his Current Positive Affirmation Color. Confusion

Index is the shift in one's current Least Favorite Color and Current Negative Affirmation Color. For a person born before 4 am: consider DD Value=DD−1, and for a Person Born after 4:01 am: no change in DD Value.

Following are some of the in-house derived formulas to calculate said mental parameters through which it is possible to accurately calculate psychological shift in a user:
1. Alignment Index (Positive Affirmation (Variable B)−Destiny Number (Variable C))×11/(A+B+C+D+E+F+G+H+I+J) (Read Values=1+2+3+4+5+6+7+8+9+10)

Formula Reduced down to: (Variable B−Variable C)×11/55

Here 11 is the Normalization Factor
2. Conditioning Index (Most Favorite Color (Variable A)−Destiny Number (Variable C))×11/(A+B+C+D+E+F+G+H+I+J) (Read Values=1+2+3+4+5+6+7+8+9+10)

Formula Reduced down to: (Variable A-Variable C)×11/55

Here 11 is the Normalization Factor
3. Dispersion Index (Most Favorite Color (Variable A)−Positive Affirmation (Variable B))×11/(A+B+C+D+E+F+G+H+I+J) (Read Values=1+2+3+4+5+6+7+8+9+10)

Formula Reduced down to: (Variable A−Variable B)×11/55

Here 11 is the Normalization Factor
4. Confusion Index (Least Favorite Color (Variable D)−Negative Affirmation (Variable E))×11/(A+B+C+D+E+F+G+H+I+J) (Read Values=1+2+3+4+5+6+7+8+9+10)

Formula Reduced down to: (Variable D×Variable E)×11/55

Here 11 is the Normalization Factor
6. Affirmation Index

(Positive Affirmation (Variable B)−Negative Affirmation (Variable E))×11/(A+B+C+D+E+F+G+H+I+J) (Read Values=1+2+3+4+5+6+7+8+9+10)

Formula Reduced down to: (Variable B−Variable E)×11/55

Here 11 is the Normalization Factor

Further, the disclosed system facilitates an individual who has a precise idea about his/her emotional state and to quantitatively judge how emotionally deviated he is at any point of time in his life and accordingly change his daily routine or habits to progress toward attaining an optimal state of mind. Further, the disclosed system helps the individual in analyzing the change in the mental state in any current situation.

Further, the present disclosure describes a system and method to evaluate a psychological shift in a user.

Further, the present disclosure describes a system and method to evaluate the change in a person's mental state as compared to their Optimum Mental State.

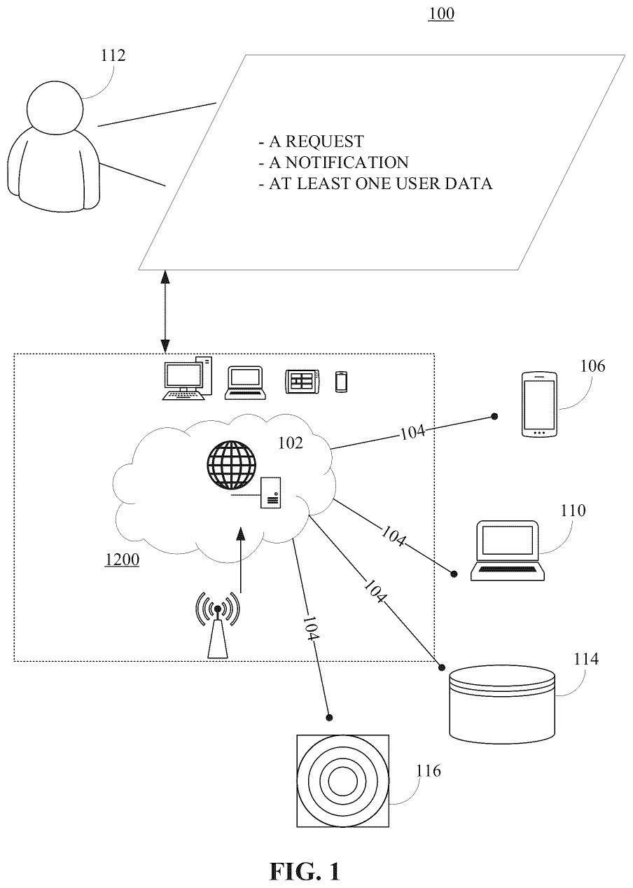

FIG. 1 is an illustration of an online platform 100 consistent with various embodiments of the present disclosure. By way of non-limiting example, the online platform 100 to facilitate evaluating psychological shifts in users may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, sensors 116, and actuators (not shown) over a communication network 104, such as, but not limited to, the Internet. Further, users of the online platform 100 may include relevant parties such as, but not limited to, end-users, individuals, users, and administrators. Accordingly, in some instances, electronic devices operated by the one or more relevant parties may be in communication with the online platform 100.

A user 112, such as the one or more relevant parties, may access the online platform 100 through a web-based software application or browser. The web-based software application may be embodied as, for example, but not be limited to, a website, a web application, a desktop application, and a mobile application compatible with a computing device 1200.

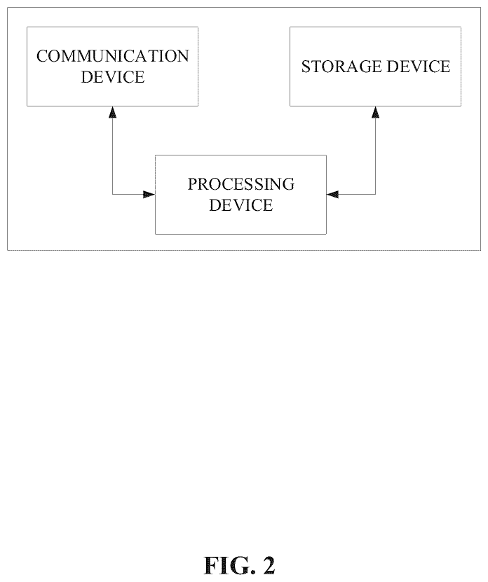

FIG. 2 is a block diagram of a system for facilitating evaluating psychological shifts in users, in accordance with some embodiments. Accordingly, the system may include a communication device, a processing device, and a storage device.

Further, the communication device may be configured for receiving a request associated with at least one user from at least one user device. Further, the communication device may be configured for transmitting one of at least one instruction and one of a plurality of color options corresponding to one of the at least one instruction to the at least one user device. Further, the communication device may be configured for receiving one of at least one selection of at least one color option from one of the plurality of color options corresponding to one of the at least one question from the at least one user device. Further, the communication device may be configured for transmitting a notification to the at least one user device.

Further, the processing device may be communicatively coupled with the communication device. Further, the processing device may be configured for determining one of at least one value corresponding to one of the at least one selection of the at least one color option based on one of the at least one selection of the at least one color option. Further, the processing device may be configured for analyzing one of the at least one value and at least one ideal value based on at least one formula. Further, the processing device may be configured for calculating at least one mental parameter of the at least one user based on the analyzing of one of the at least one value and the at least one ideal value. Further, the processing device may be configured for determining a psychological shift in the at least one user from the ideal psychological state based on the at least one mental parameter. Further, the processing device may be configured for generating the notification of the psychological shift of the at least one user based on the determining of the psychological shift.

Further, the storage device may be communicatively coupled with the processing device. Further, the storage device may be configured for retrieving the at least one ideal value corresponding to an ideal psychological state of the at least one user based on the request.

Further, in some embodiments, the at least one user device may include at least one first sensor. Further, the at least one first sensor may be configured for generating one of the at least one selection of the at least one color option from one of the plurality of color options based on detecting at least one of a physiological response and an emotional response of the at least one user. Further, the physiological response may include a change in heartbeat, etc. Further, the emotional response may include a feeling of happiness, a feeling of sadness, etc.

Further, in some embodiments, the communication device may be configured for receiving at least one user data associated with at least one user from at least one user device. Further, the processing device may be configured for analyzing the at least one user data. Further, the processing device may be configured for generating the at least one ideal value corresponding to the ideal psychological state of the at least one user based on the analyzing of the at least one user data. Further, the storage device may be configured for storing the at least one ideal value corresponding to the ideal psychological state of the at least one user.

Further, in an embodiment, the at least one user data may include birth data of the at least one user. Further, the birth data may include a date of birth, a place of birth, etc. Further, the analyzing of the at least one user data may include analyzing the birth data using at least one first formula. Further, the generating of the at least one ideal value may be based on the analyzing of the birth data using the at least one first formula.

Further, in an embodiment, the communication device may be configured for transmitting at least one message to the at least one user device. Further, the at least one message instructs the at least one user to imagine the ideal psychological state of the at least one user for at least one duration. Further, the at least one user device may include at least one sensor. Further, the at least one sensor may be configured for generating the at least one user data based on detecting at least one of a physiological state and an emotional state of the at least one user during the at least one duration. Further, the physiological state may include a heartbeat rate, etc. Further, the emotional state may include happiness, sadness, anger, etc.

Further, in an embodiment, the analyzing of the at least one user data may include analyzing the at least one user data using at least one machine learning model. Further, the at least one machine learning model may be trained using at least one of a plurality of physiological states and a plurality of emotional states and a plurality of corresponding ideal values. Further, the generating of the at least one ideal value may be based on the analyzing of the at least one user data using the at least one machine learning model.

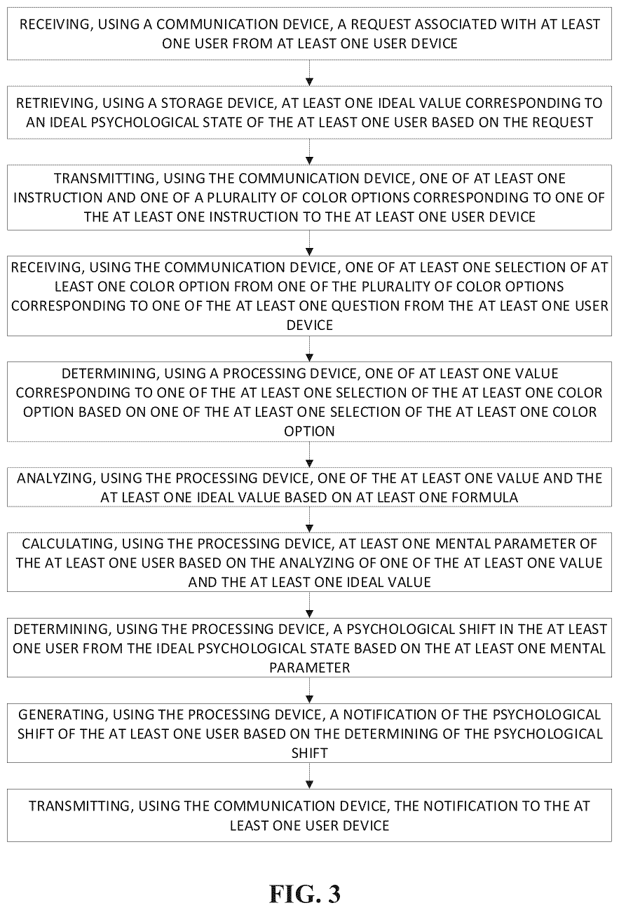

FIG. 3 is a flowchart of a method for facilitating evaluating psychological shifts in users, in accordance with some embodiments. Accordingly, the method may include receiving, using a communication device, a request associated with at least one user from at least one user device. Further, the at least one user may include at least one client, at least one individual, at least one person, etc. Further, the at least one user device may include at least one client device, at least one computing device, etc.

Further, the method may include a step of retrieving, using a storage device, at least one ideal value corresponding to an ideal psychological state of the at least one user based on the request. Further, the at least one ideal value may include an epistemic number, a destiny number, etc. of the at least one user

Further, the method may include a step of transmitting, using the communication device, one of at least one instruction and one of a plurality of color options corresponding to one of the at least one instruction to the at least one user device. Further, one of the at least one instruction may include a direction for selecting a color under at least one criterion. Further, one of the at least one instruction may be associated with at least one variable. Further, the at least one variable may include most favorite color, positive affirmation, least favorite color, negative affirmation, person's mood, art selection, activity selection, psychological profile, Vritti, etc.

Further, the method may include a step of receiving, using the communication device, one of at least one selection of at least one color option from one of the plurality of color options corresponding to one of the at least one question from the at least one user device.

Further, the method may include a step of determining, using a processing device, one of at least one value corresponding to one of the at least one selection of the at least one color option based on one of the at least one selection of the at least one color option. Further, the at least one value may include a numerical value.

Further, the method may include a step of analyzing, using the processing device, one of the at least one value and the at least one ideal value based on at least one formula.

Further, the method may include a step of calculating, using the processing device, at least one mental parameter of the at least one user based on the analyzing of one of the at least one value and the at least one ideal value. Further, the at least one mental parameter may include a coefficient of an Alignment index, a Conditioning index, a Dispersion Index, a Confusion index, etc.

Further, the method may include a step of determining, using the processing device, a psychological shift in the at least one user from the ideal psychological state based on the at least one mental parameter.

Further, the method may include a step of generating, using the processing device, a notification of the psychological shift of the at least one user based on the determining of the psychological shift.

Further, the method may include a step of transmitting, using the communication device, the notification to the at least one user device.

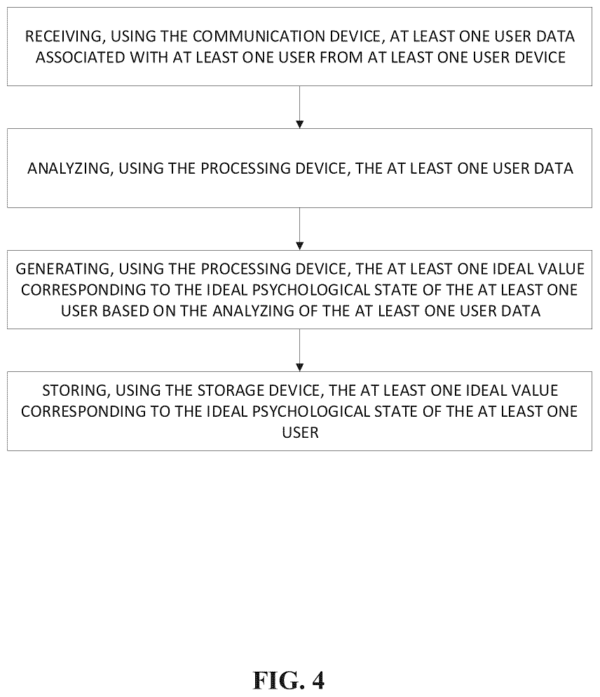

FIG. 4 is a flowchart of a method of determining ideal values of ideal psychological states of the users for facilitating the evaluating of the psychological shifts in the users, in accordance with some embodiments. Accordingly, the method may include a step of receiving, using the communication device, at least one user data associated with at least one user from at least one user device. Further, the at least one user data may include birth data of the at least one user. Further, the birth data may include a date of birth, a place of birth, etc.

Further, the method may include a step of analyzing, using the processing device, the at least one user data.

Further, the method may include a step of generating, using the processing device, the at least one ideal value corresponding to the ideal psychological state of the at least one user based on the analyzing of the at least one user data.

Further, the method may include a step of storing, using the storage device, the at least one ideal value corresponding to the ideal psychological state of the at least one user.

FIG. 5 illustrates a graphical interface screen for receiving a selection for a variable A for facilitating the evaluating of the psychological shifts in the users, in accordance with some embodiments.

FIG. 6 illustrates a graphical interface screen for receiving a selection for a variable B for facilitating the evaluating of the psychological shifts in the users, in accordance with some embodiments.

FIG. 7 illustrates a graphical interface screen for receiving a selection for a variable D for facilitating the evaluating of the psychological shifts in the users, in accordance with some embodiments.

FIG. 8 illustrates a graphical interface screen for receiving a selection for a variable E for facilitating the evaluating of the psychological shifts in the users, in accordance with some embodiments.

FIG. 9 illustrates a graphical interface screen for receiving a selection for a variable A1 for facilitating the evaluating of the psychological shifts in the users, in accordance with some embodiments.

FIG. 10 is a table for values for selections of variables for facilitating the evaluating of the psychological shifts in the users, in accordance with some embodiments.

FIG. 11 is a table for values for selections of other variables (variable A1) for facilitating the evaluating of the psychological shifts in the users, in accordance with some embodiments.

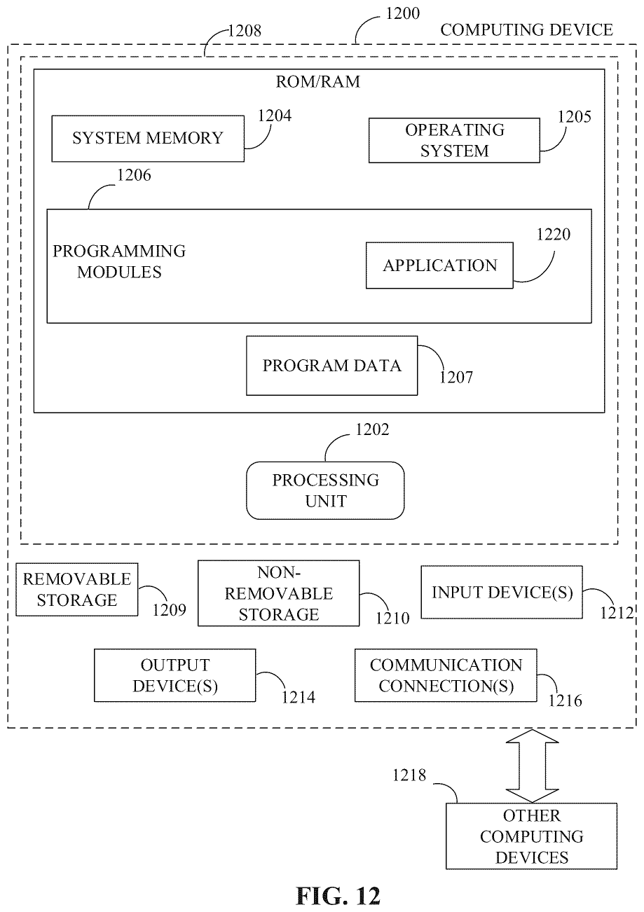

With reference to FIG. 12, a system consistent with an embodiment of the disclosure may include a computing device or cloud service, such as computing device 1200. In a basic configuration, computing device 1200 may include at least one processing unit 1202 and a system memory 1204. Depending on the configuration and type of computing device, system memory 1204 may comprise, but is not limited to, volatile (e.g. random-access memory (RAM)), non-volatile (e.g. read-only memory (ROM)), flash memory, or any combination. System memory 1204 may include operating system 1205, one or more programming modules 1206, and may include a program data 1207. Operating system 1205, for example, may be suitable for controlling computing device 1200's operation. In one embodiment, programming modules 1206 may include image-processing module, machine learning module and/or image classifying module. Furthermore, embodiments of the disclosure may be practiced in conjunction with a graphics library, other operating systems, or any other application program and is not limited to any particular application or system. This basic configuration is illustrated in FIG. 12 by those components within a dashed line 1208.

Computing device 1200 may have additional features or functionality. For example, computing device 1200 may also include additional data storage devices (removable and/or non-removable) such as, for example, magnetic disks, optical disks, or tape. Such additional storage is illustrated in FIG. 12 by a removable storage 1209 and a non-removable storage 1210. Computer storage media may include volatile and nonvolatile, removable and non-removable media implemented in any method or technology for storage of information, such as computer-readable instructions, data structures, program modules, or other data. System memory 1204, removable storage 1209, and non-removable storage 1210 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1200. Any such computer storage media may be part of device 1200. Computing device 1200 may also have input device(s) 1212 such as a keyboard, a mouse, a pen, a sound input device, a touch input device, a location sensor, a camera, a biometric sensor, etc. Output device(s) 1214 such as a display, speakers, a printer, etc. may also be included. The aforementioned devices are examples and others may be used.

Computing device 1200 may also contain a communication connection 1216 that may allow device 1200 to communicate with other computing devices 1218, such as over a network in a distributed computing environment, for example, an intranet or the Internet. Communication connection 1216 is one example of communication media. Communication media may typically be embodied by computer-readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer-readable media as used herein may include both storage media and communication media.

As stated above, a number of program modules and data files may be stored in system memory 1204, including operating system 1205. While executing on processing unit 1202, programming modules 1206 (e.g., application 1220 such as a media player) may perform processes including, for example, one or more stages of methods, algorithms, systems, applications, servers, databases as described above. The aforementioned process is an example, and processing unit 1202 may perform other processes. Other programming modules that may be used in accordance with embodiments of the present disclosure may include sound encoding/decoding applications, machine learning application, acoustic classifiers, etc.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general-purpose graphics processor-based systems, multiprocessor systems, microprocessor-based or programmable consumer electronics, application-specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer-readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid-state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the disclosure has been explained in relation to its preferred embodiment, it is to be understood that many other possible modifications and variations can be made without departing from the spirit and scope of the disclosure.

1. A method for evaluating psychological shifts in users, comprising:
receiving, by a communication device, a request associated with at least one user from at least one user device;
retrieving, by a storage device, at least one ideal value corresponding to an ideal psychological state of the at least one user based on the request;
transmitting, by the communication device, one of at least one instruction and one of a plurality of color options corresponding to the at least one instruction to the at least one user device;
receiving, by the communication device, at least one selection of at least one color option from one of the plurality of color options corresponding to one of at least one question from the at least one user device;
determining, by a processing device, at least one value corresponding to one of the at least one selection of the at least one color option based on one of the at least one selection of the at least one color option;
analyzing, by the processing device, the at least one value and the at least one ideal value based on at least one formula;
calculating, by the processing device, at least one mental parameter of the at least one user based on the analyzing the at least one value and the at least one ideal value;
determining, by the processing device, a psychological shift in the at least one user from the ideal psychological state based on the at least one mental parameter;
generating, by the processing device, a notification of the psychological shift of the at least one user based on the determining of the psychological shift; and
transmitting, by the communication device, the notification to the at least one user device.

2. The method of claim 1, wherein the at least one formula is used to calculate the mental parameter, where the at least one formula comprise an Alignment Index, a Conditioning Index, a Dispersion Index, a Confusion Index, an Affirmation Index, or any combination thereof.

3. A system for evaluating psychological shifts in users, comprising:
a communication device configured to:
receive a request associated with at least one user from at least one user device, transmit one of at least one instruction and one of a plurality of color options corresponding to one of the at least one instruction to the at least one user device,
receive at least one selection of at least one color option from one of the plurality of color options corresponding to one of at least one question from the at least one user device, and transmit a notification to the at least one user device;
a processing device communicatively coupled with the communication device, configured to:
determine one of at least one value corresponding to one of the at least one selection of the at least one color option based on one of the at least one selection of the at least one color option,
analyze one of the at least one value and at least one ideal value based on at least one formula, calculate at least one mental parameter of the at least one user based on the analyzing of one of the at least one value and the at least one ideal value,
determine a psychological shift in the at least one user from an ideal psychological state based on the at least one mental parameter, and generate the notification of the psychological shift of the at least one user based on the determining of the psychological shift;
a storage device communicatively coupled with the processing device, configured to store at least one ideal value corresponding to an ideal psychological state of the at least one user based on the request and provide the at least one ideal value to the processing device.

4. The system of claim 3, wherein the communication device is comprises a smartphone, tablet, computer, a wearable device, or any combination thereof.

5. The system of claim 3, wherein the plurality of color options are determined based on a color psychology model.

6. The system of claim 3, wherein the at least one instruction includes a sequence of questions designed to assess specific aspects of the psychological state of the at least one user.

7. The system of claim 3, wherein the at least one formula is derived from a psychological evaluation model.

8. The system of claim 3, wherein the processing device uses machine learning algorithms to analyze the at least one value and the at least one ideal value.

9. The system of claim 3, wherein the processing device is configured to analyze historical data from the user to determine the psychological shift.

10. The system of claim 3, wherein the processing device is configured to compare the at least one mental parameter to a normative database of mental parameters.

11. The system of claim 3, wherein the notification of the psychological shift includes recommendations for actions the user can take to address the psychological shift.

12. The system of claim 3, further comprising a user interface on the communication device that allows the user to interact with the system and input data.

13. The system of claim 3, wherein the communication device is configured to receive input from additional devices, such as biometric sensors, to enhance the evaluation of the psychological state of the at least one user.

14. The system of claim 3, wherein the system is configured to provide periodic assessments and notifications to track the psychological state of the at least one user over time.

15. The system of claim 3, wherein the system is configured to allow users to set personal goals related to the user's psychological state, and provide feedback based on progress towards those goals.

16. The system of claim 3, wherein the system is configured to integrate with other mental health applications or services to provide a comprehensive approach to psychological well-being.

17. The system of claim 3, wherein the system is configured to provide anonymized data to researchers or healthcare providers to improve understanding of psychological shifts and mental health.

18. The system of claim 3, wherein the communication device is configured to provide personalized feedback to the user based on an individual psychological state of the at least one user and ideal values.

19. The system of claim 3, wherein the processing device is configured to generate a visual representation of the psychological state of the at least one user based on the analysis of the at least one mental parameter.

20. The system of claim 3, wherein the storage device is configured to store data related to the psychological state of the at least one user, including historical data, ideal values, and progress towards personal goals, to provide a comprehensive overview of a mental well-being of the at least one user.
